(19) United States
(12) Patent Application Publication
(10) Pub. No.: US 2010/0233767 A1
(43) Pub. Date: Sep. 16, 2010

(54) PROCESS FOR THE RECOVERY OF MAGNESIUM FROM A SOLUTION AND PRETREATMENT

(76) Inventor: David McMurran, Cherrybrook (AU)

Correspondence Address: BERLINER & ASSOCIATES, 555 WEST FIFTH STREET, 31ST FLOOR, LOS ANGELES, CA, 90013, US

(21) Appl. No.: 12/666,469

(22) PCT Filed: Jun. 30, 2008
(86) PCT No.: PCT/AU08/00958

§ 371 Date: May 27, 2010

(30) Foreign Application Priority Data
Jun. 28, 2007 (AU) 2007903488

(51) Int. Cl. C12P 7/64; C01F 5/14; C22B 26/22; C22B 4/02; C22B 3/00

(52) U.S. Cl. 435/134; 423/635; 75/596; 75/10.19

(57) ABSTRACT

A process for the recovery of magnesium from a solution containing soluble magnesium, the process comprising, precipitating magnesium hydroxide from the solution, forming an oxide blend including magnesium oxide derived from the precipitated magnesium hydroxide together with calcium oxide, reducing the oxide blend to form a magnesium metal vapour and condensing the vapour to recover magnesium metal.

### FIELD OF THE INVENTION

The present invention is generally concerned with a process for the recovery of magnesium metal and an associated pretreatment process. More particularly, the invention relates to processes involving the precipitation of magnesium hydroxide from a solution containing soluble magnesium. The pretreatment process, which may also be employed independently of the process for recovery of magnesium, takes advantage of sodium chloride present in the solution as a source for the production of sodium hydroxide that may be used in the precipitation of the magnesium hydroxide from the solution.

### BACKGROUND TO THE INVENTION

Magnesium is found in over 60 minerals, although only dolomite, magnesite, brucite, carnallite, talc, and olivine are of commercial importance. In the United States this metal is principally obtained by electrolysis of fused magnesium chloride from brines, wells, and sea water:

cathode: Mg²⁺+2 e⁻→Mg

anode: 2 Cl⁻→Cl_{2 (gas)}+2 e⁻

The United States has traditionally been the major world supplier of this metal, supplying 45% of world production even as recently as 1995. Today, the US market share is at 7%, with a single domestic producer left, US Magnesium, a company born from now-defunct Magcorp. China has taken over as the dominant supplier, pegged at 60% world market share, which increased from 4% in 1995. Unlike the above described electrolytic process, China is almost completely reliant on a different method of obtaining the metal from its ores, the silicothermic Pidgeon process (the reduction of the oxide at high temperatures with silicon).

The Mg²⁺ cation is the second most abundant cation in seawater (occurring at about 12% of the mass of sodium there), which makes seawater and sea-salt an attractive commercial source of Mg. Historically, to extract the magnesium, calcium carbonate is added to sea water to form magnesium carbonate precipitate.

MgCl₂+CaCO₃→MgCO₃+CaCl₂

Magnesium carbonate is insoluble in water so it can be filtered out, and reacted with hydrochloric acid to obtain concentrated magnesium chloride.

MgCO₃+2HCl→MgCl₂+CO₂+H₂O

From magnesium chloride, electrolysis produces magnesium as described above.

Processes for the desalination of water are known. For example, processes have been proposed that involve the deionisation of water to produce a desalinated water product and a waste stream. In such processes, due to the substantial flows of water through the desalination plant, a substantial waste stream is produced which may be problematic and require some form of post-treatment downstream.

Currently, there also exists a dire need on a global level to reduce the amount of carbon dioxide being emitted into the atmosphere. This issue is of particular importance in the current political climate. However, to date there have been few methods proposed that are suitable for the treatment of the substantial emissions produces by, for example, a coal power plant.

The reaction of magnesium hydroxide with carbon dioxide to form magnesium carbonate is known, as follows:

Mg(OH)₂+CO₂→MgCO₃+H₂O

However, due to the flow rates involved in desalination waste streams this reaction has been considered inappropriate for the recovery of the carbonate. That is, in dealing with the issue of water treatment from such high flow sources, the reaction requires a substantial amount of carbon dioxide. Furthermore, the quantity of magnesium carbonate produced if one were to follow only this route, even though a useful commodity, has been hitherto considered to be unusable in such quantities. Still further, the above reaction is generally incomplete, converting only about 80% of the magnesium hydroxide present during the reaction.

The invention in one aspect provides a process for the recovery of magnesium from a solution containing soluble magnesium, such as in a high throughput solution feed, which may advantageously be used in conjunction with other peripheral processes. In another aspect, Applicant has devised a pretreatment process that may, or may not be used in conjunction with the process for magnesium recovery. Advantageously, the processes are used in combination to provide synergistic economical benefits.

### SUMMARY OF THE INVENTION

According to a first aspect of the invention there is provided a process for the recovery of magnesium from a solution containing soluble magnesium, the process comprising:
precipitating magnesium hydroxide from the solution;
forming an oxide blend including magnesium oxide derived from the precipitated magnesium hydroxide together with calcium oxide;
reducing the oxide blend to form a magnesium metal vapour; and
condensing the vapour to recover magnesium metal.

Precipitation of the magnesium hydroxide may be facilitated by treatment of the solution containing soluble magnesium with at least one of the group consisting of sodium hydroxide, calcium oxide, calcium magnesium oxide and calcium hydroxide. According to one embodiment, the solution containing soluble magnesium is treated with calcium oxide and/or calcium magnesium oxide to separately recover magnesium hydroxide and calcium hydroxide. The magnesium hydroxide and calcium hydroxide are preferably recombined and calcined to form the oxide blend. In an alternative embodiment, as will be dealt with below in more detail, the solution containing soluble magnesium is treated with sodium hydroxide that may be derived from the solution prior to precipitation of the magnesium hydroxide.

In a preferred embodiment, prior to the precipitation of magnesium, the solution is treated to remove sodium chloride. The sodium chloride may be removed by any suitable means. In a particular embodiment the solution is subjected to electrodialysis, for example bipolar membrane electrodialysis, to remove sodium chloride. The sodium chloride thus removed is preferably electrolysed, or otherwise treated, to form sodium hydroxide. The sodium hydroxide may advantageously be used in the precipitation of the magnesium hydroxide as described above. It will be readily appreciated the advantages provided by this cycle.

In certain embodiments, it may be advantageous to introduce a reductant to the oxide blend. Preferably, the reductant is ferro silicon or aluminium oxide, although other known reductants may also be suitable. Preferably, aluminium oxide is used as the reductant.

The oxide blend is reduced, preferably in a furnace such as a plasma DC Arc furnace, to produce magnesium metal vapour. Other known furnaces may also be suitable. In a particular embodiment, aluminium metal is added to the oxide blend before and/or during the reduction process.

The temperature used during the reduction process will be somewhat dependent on the reductanct employed. Generally, the temperature is maintained at from 1100° C. to 1700° C. during the reduction process. The lower temperatures within this range are considered more appropriate when the reductant is aluminium oxide, whilst the higher temperatures within this range are considered more appropriate when the reductant is ferro silicon.

According to this aspect of the invention it may be desirable to convert a proportion of the magnesium hydroxide to magnesium carbonate in a carbon dioxide sequestration process. That is, if desired, carbon dioxide may be reacted with the precipitated magnesium hydroxide to form magnesium carbonate. This may, in certain circumstances, mitigate issues relating to carbon dioxide emission into the atmosphere.

If carbon dioxide is to be reacted with the magnesium hydroxide and the magnesium hydroxide is present in solution as micron sized particles, the magnesium hydroxide is preferably reacted with the carbon dioxide in a fluidised bed reactor. More particularly, the magnesium hydroxide and carbon dioxide are advantageously mixed in the fluidised bed reactor in a ratio of 1.3:1. Generally, the reaction in the fluidised bed reactor is conducted at a temperature of from about 400° C. to 420° C. for a period of up to about 30 minutes.

At least a portion of the magnesium hydroxide may, in certain embodiments, be reacted with super critical carbon dioxide. Super critical carbon dioxide is defined as that which is at 31° C. and 80 atmospheres. According to a particular embodiment, the magnesium hydroxide is reacted with the supercritical carbon dioxide in aqueous phase solution in a fluidised bed reactor.

In certain embodiments, at least a portion of the solution containing soluble magnesium is treated with supercritical carbon dioxide to cause precipitation of magnesium carbonate and calcium carbonate. Following treatment with the super critical carbon dioxide a by-product stream from the solution containing soluble magnesium may be treated by centrifuging, flocculation and/or chemical dosing to recover valuables.

According to certain embodiments where carbon dioxide is reacted with the magnesium hydroxide, NOₓ and SOₓ are separated from the carbon dioxide prior to the reaction of the carbon dioxide with the magnesium hydroxide. In that case, an ammonia by-product is preferably collected after NOₓ and SOₓ separation and combined with at least a portion of the magnesium carbonate product to form an ammonia-based fertiliser.

If desired, in certain embodiments of the invention a portion of the solution containing soluble magnesium may be treated with carbon dioxide to form a sodium carbonate by-product. In that case, the ammonia by-product collected following NOₓ and SOₓ separation may be reacted with the sodium carbonate by-product. That is, use of ammonia from post carbon capture (PCC) may be possible in accordance with certain embodiments of the invention. As will be known, ammonia is a waste product from PCC processing conducted by power stations and other industries. Use of such a waste product provides advantages in terms of both economics and environmental concerns.

Advantageously, it has been determined that a valuable peripheral process may provide for a synergistic relationship with the process described above. Particularly, it is envisaged that salt, in the form of sodium chloride, recovered from the process or entrained in a waste stream emitted from the process may be utilised in a bioreactor, for example a photo-bioreactor, for the production of biomass oil that may be subsequently processed to biofuel. Likewise, it is envisaged that carbon dioxide produced during processing, or otherwise produced, may be similarly utilised. Therefore, in a particularly preferred embodiment sodium chloride recovered from the process and/or entrained in a waste stream emitted from the process and carbon dioxide are fed to a bioreactor housing microalgae for the production of biomass oil.

In a particular embodiment, treatment of the solution containing soluble magnesium includes an electrodialysis process wherein a plurality of streams having different salinity (i.e. salt content) are emitted and fed to separate bioreactors, each housing microalgae. In this embodiment, each stream emitted from the process may be manipulated to a desired salinity dependent on the microalgae housed in the respective bioreactor to which the stream is being fed. To that end, it will be appreciated that different microalgaes may facilitate the production of different forms of fuel and the process of the invention including this synergistic peripheral process may therefore be tailored as desired.

Microalgae may also be located in a pond into which the waste stream is fed prior to being fed to the bioreactor. As such, the microalgae may be grown in the pond for subsequent transferral to the bioreactor where it is converted to biomass. Additional nutrients may also be fed to the pond and/or bioreactor as desired. Such nutrients may be by-products of the process of the invention, or may be derived from an external source.

The microalgae may be harvested as desired for subsequent refining to biofuel. In that regard, it will be understood that the specie of algae is not particularly limited. Those with high oil content are particularly preferred. For example, Chlorella and Spirulina species have been found to be capable of producing more than 30 times the amount of oil (per year per unit of land area) when compared to oil seed crops.

As will be discussed in more detail below, vapour pressure within the furnace is advantageously controlled to ensure volumetric flows of vapour are aligned and directed towards an exit port for the furnace.

It is believed that the above described invention will find particular application in the treatment of waste streams from desalination processes.

To that end, according to a particular aspect of the invention there is provided a process for the recovery of magnesium from a waste stream of a desalination plant, the process comprising:
treating the waste stream using electrodialysis to remove sodium chloride;
converting the sodium chloride removed from the waste stream to sodium hydroxide;
reacting the sodium hydroxide with soluble magnesium in the waste stream to precipitate magnesium hydroxide;
forming an oxide blend including magnesium oxide derived from the precipitated magnesium hydroxide together with calcium oxide;
reducing the oxide blend to form magnesium metal vapour;
condensing the magnesium metal vapour to recover magnesium metal; and
reporting at least a portion of the sodium chloride removed from the waste stream and carbon dioxide to a bioreactor housing microalgae for the production of biofuel.

Additional features and embodiments of the invention relevant to this particular aspect of the invention will be readily apparent from the above discussion of the invention.

In arriving at the invention, Applicant has also arrived at an advantage pretreatment process that may be independently employed, but which provides specific advantages when used in conjunction with the above described process. The pretreatment process, which is also briefly described above, finds particular advantage in the treatment of waters having high salinity, such as those emitted from desalination plants. With this example, waste from desalination plants is in many instances reported out to the open ocean, which may have an environmental impact on sea life at the waste outlet. In particular, it is recognised that the emission of high salinity waste into the ocean generally increases acidity of the water at the waste outlet. The pretreatment process devised by Applicant aims at alleviating the environmental impact of waste emission from desalination plants into the ocean, and which advantageously provides a source of magnesium hydroxide which may be used in the process of the first aspect of the invention, or that may be otherwise used.

According to a second aspect of the invention there is provided a method for treatment of a waste stream emitted from a desalination plant comprising:
treating the waste stream to remove sodium chloride;
converting the sodium chloride removed from the waste stream to sodium hydroxide; and
reacting at least a portion of the sodium hydroxide with soluble magnesium in the waste stream to precipitate magnesium hydroxide.

Preferably, the process of the second aspect of the invention also includes reporting at least a portion of the sodium hydroxide back to the waste stream and/or to an outlet for the waste stream thereby modulating the pH of the water at the outlet for the waste stream.

As was the case for embodiments of the first aspect of the invention, the treatment of the waste stream to remove sodium chloride preferably includes electrodialysis of the waste stream, for example using bipolar membrane electrodialysis. Advantageously, the waste stream is treated in a single pass to remove sodium chloride.

Likewise, the sodium chloride is preferably converted to sodium hydroxide using electrolysis, as described above in relation to embodiments of the first aspect of the invention.

### DETAILED DESCRIPTION OF THE INVENTION

A more detailed description of the invention will now be provided with reference to the accompanying drawings. It will be appreciated that the drawings are provided for exemplification only and should not be construed as limiting on the invention in any way. It will also be appreciated that various side processing options and by-product recirculation routes are not illustrated in the drawings. Such additional options and routes are, however, within the ambit of the present invention. Referring to the drawings:

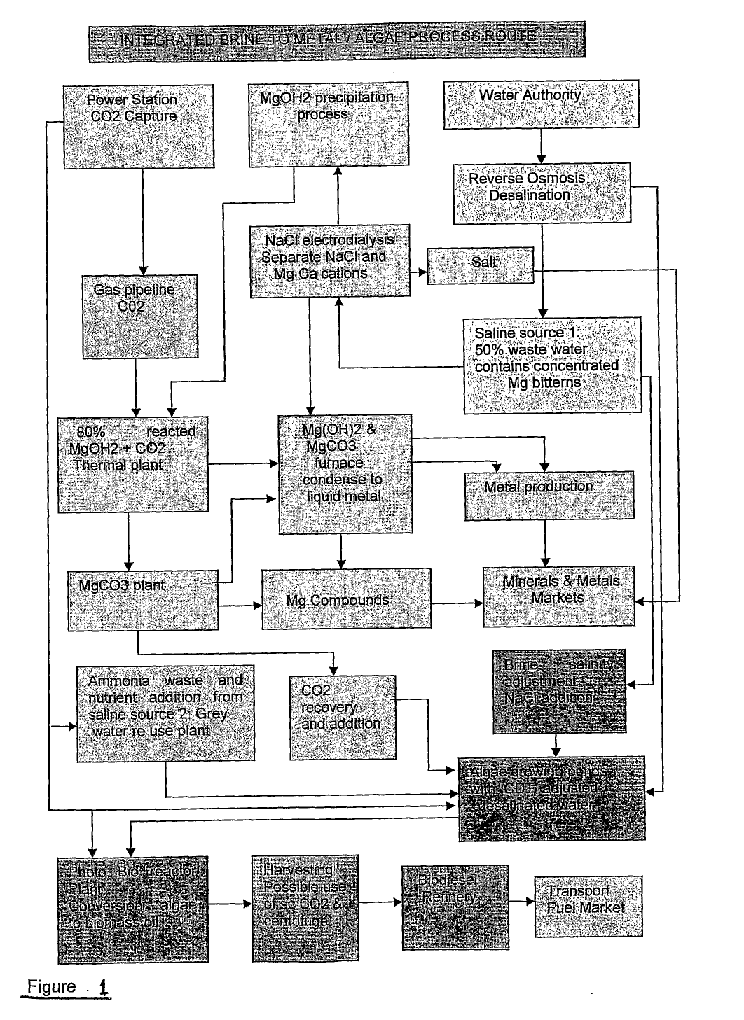

FIG. 1 is a flow chart illustrating a combined processing route that integrates the process for the recovery of magnesium metal from a solution with other peripheral processes, including a process for the production of biodiesel employing microalgae which takes advantage of output from the metal recovery process;

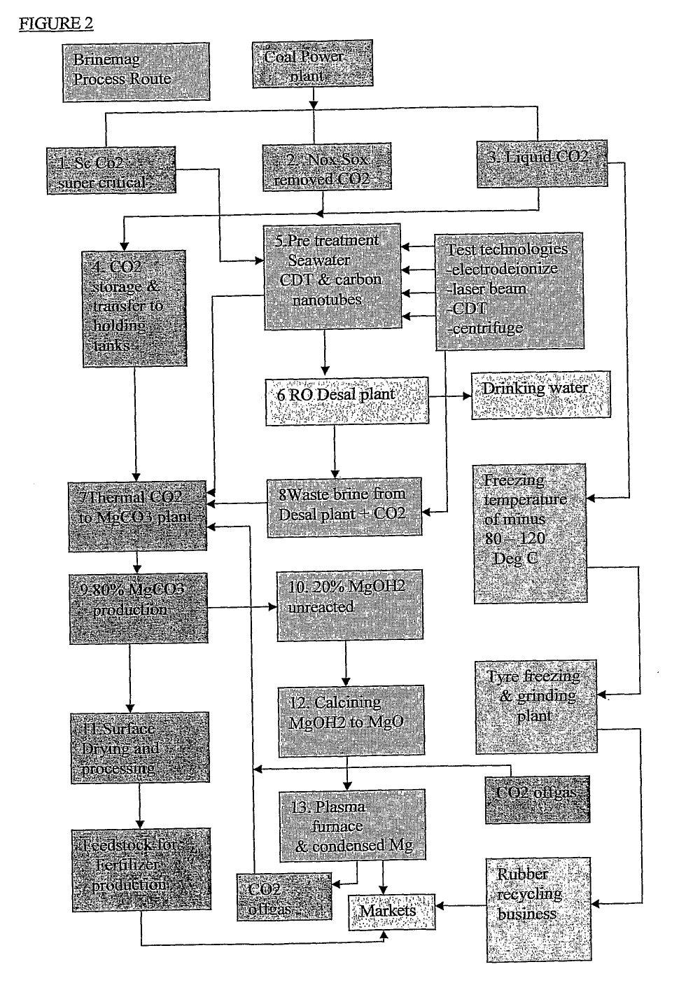

FIG. 2 is a flow chart illustrating a combined processing route for carbon dioxide carbonation and magnesium metal recovery in accordance with certain embodiments of the invention;

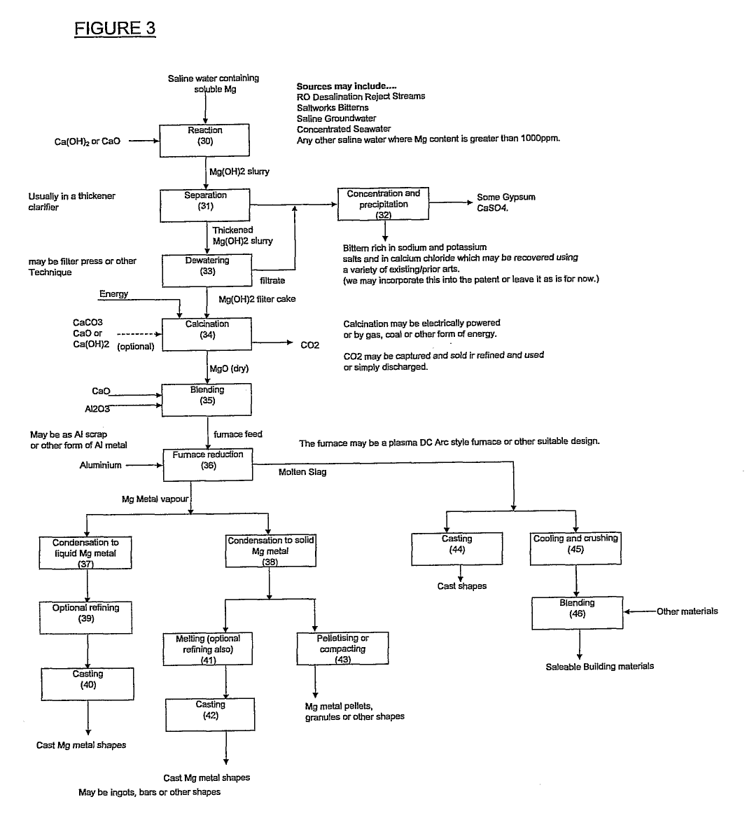

FIG. 3 is a flow chart illustrating a processing route for magnesium metal recovery, in more detail, in accordance with certain embodiments of the invention;

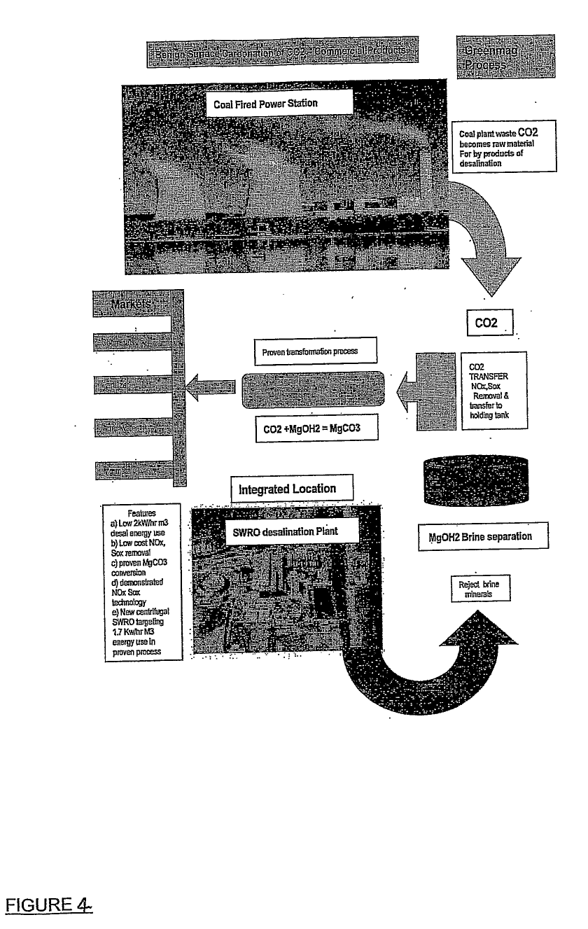

FIG. 4 is flow chart illustrating a processing route incorporating the treatment of carbon dioxide from a coal-fired power station and a waste stream from a desalination plant.

Referring to FIG. 1, a fully integrated processing route is illustrated. The processing route provides substantial advantages through utilisation of various by-product and peripheral process to a central metal recovery process. The synergies provided through the integration of the various processes are described in detail below.

As will be appreciated from FIG. 1, a waste stream from a reverse osmosis desalination process containing concentrates magnesium bitterns is sourced. The waste solution is treated using electrodialysis to separate sodium chloride from magnesium and calcium cations. Whilst it is not intended to discuss the electrodialysis process in substantial detail here, it is envisaged that this process may advantageously include bipolar membrane electrodialysis. This process, also coined “Water Splitting”, converts aqueous salt solutions into acids and bases without chemical addition. It is an electrodialysis process since ion exchange membranes are used to separate ionic species in solution with the driving force of an electrical field, but it is different by the unique water splitting capability of the bipolar membrane. In addition, the process offers unique opportunities to directly acidify or basify process streams without adding chemicals, avoiding by-product or waste streams and costly downstream purification steps.

Under the driving force of an electrical field, a bipolar membrane can efficiently dissociate water into hydrogen (H+, in fact “hydronium” H3+) and hydroxyl (OH−) ions. It is formed of an anion- and a cation-exchange layer that are bound together, either physically or chemically, and a very thin interface where the water diffuses from the outside aqueous salt solutions. The transport out of the membrane of the H+ and OH− ions obtained from the water splitting reaction is possible if the bipolar membrane is oriented correctly (there is no current reversal in water splitting). With the anion-exchange side facing the anode and the cation-exchange side facing the cathode, the hydroxyl anions will be transported across the anion-exchange layer and the hydrogen cations across the cation-exchange layer. Therefore, a bipolar membrane allows the efficient generation and concentration of hydroxyl and hydrogen ions at its surface (up to 10N). These ions are used in an electrodialysis stack to combine with the cations and anions of the salt to produce acids and bases.

A good bipolar membrane has a strong, permanent bond between the two layers and a thin interface to reduce the voltage drop. It also allows the water to easily diffuse inside to the interface and feed the water splitting reaction so that a high current density can be applied to minimize the required membrane area.

Sodium chloride recovered from the electrodialysis process is converted to sodium hydroxide which is used to precipitate magnesium hydroxide in a precipitation process. The magnesium hydroxide precipitated may be used as a feedstock for reaction with carbon dioxide to precipitate magnesium carbonate, or may be fed directly to a furnace for reduction to a magnesium metal vapour and subsequent condensing to the liquid metal form.

If carbon dioxide is used to convert a portion of the magnesium hydroxide to magnesium carbonate, the carbonate form may used as a base stock for the production of magnesium compounds which may be marketed. It is also envisage that in some instances the carbonate form may be directed to the furnace, again for reduction and subsequent condensing to liquid magnesium metal.

An integrated biodiesel production route is also illustrated in FIG. 1. As will be appreciated from the Figure, several by-products from the integrated process may advantageously be employed providing synergies that result in substantial economic and environmental benefits.

In particular, sodium chloride sourced from the original waste stream is advantageously used as a feed for algae growing ponds containing microalgae. The salinity of the feed may be adjusted as desired depending on the nature of the microalgae being used. Likewise, carbon dioxide recovered from the process in various manners may be fed to the growing ponds as desired, as may waste and nutrients recovered in cases where carbon dioxide is captured from a power station and treated.

Turning to the biodiesel recovery process, microalgae is advantageously transferred to a photo bioreactor plant where it is used to form biomass oil. Microalgae is subsequently harvested, possible using super critical carbon dioxide, which may also be sourced from the fully integrated process, and centrifuging.

Referring to FIG. 2, a detailed illustration of a process involving the combined carbonation or sequestration of carbon dioxide and formation of magnesium metal is shown.

Referring firstly to the carbonation process, carbon dioxide generated from a coal power plant may be used in the process in three different forms. They are supercritical carbon dioxide (Sc CO₂), gaseous CO₂ and liquid CO₂.

Sc CO₂ (1) may be fed directly to seawater in a pretreatment stage (5) where Mg, Ca and Na may be extracted by pressure drop from the solution, after which Sc CO₂ continues on separately. This procedure may be used to quickly extract the minerals which may then be separately centrifuged. In an alternative embodiment, the seawater is mixed with Sc CO₂ to produce MgCO₃.

Gaseous CO₂ (2) may be stored (4) or used directly. Generally, NOₓ and SOₓ are removed from the gaseous CO₂ (2) before reaction with Mg(OH)₂. This produces an ammonia by-product that may be suitably employed in later processing, as will be discussed in more detail below. When using gaseous CO₂ (2), Mg(OH)₂ is extracted by centrifuge processing and calcining to 10-20 micron size. The CO₂ and Mg(OH)₂ are reacted in a heated chamber, typically a fluidised bed reactor, at a temperature generally of less than 410° C. This rapidly converts about 80% of the Mg(OH)₂ to MgCO₃. The remaining 20% is captured for use in the metal recovery process as discussed in more detail below.

Liquid CO₂ (3) may be stored (4), prior to reaction with the Mg(OH)₂. The CO₂ in liquid form and Mg(OH)₂ are reacted at high temperature and pressure to sequester CO₂ to magnesium carbonate. The liquid CO₂ is in a form that may interact with powdered CO₂ in aqueous suspension at ambient temperature.

A portion of the liquid CO₂ is used in a side process that involves tyre freezing and grinding (as shown). The granulated rubber that is produced is recycled for future use as desired.

Pretreatment (5) of the seawater may be included. This may employ the use of deionisation processes and/or carbon nanotube filtration to remove impurities, such as chloride or other halides, from the seawater. Such processes may also be used for algae removal if desired, which may result in faster flows. In that case, waste brine produced may be reticulated for reaction with CO₂. The pretreatment may also employ electrodialysis to separate magnesium, calcium and sodium ions from the seawater as described above.

Rehydration, flocculation and cooling may be used to remove calcium, potassium and boron inclusions. Centrifuging may then be adopted to separate magnesium, calcium and potassium as products, or for future use.

In the desalination plant (6), treated water is produced and fed off in a stream for drinking or other uses (as shown). A waste brine (8) from the desalination plant (6) is treated by addition of lime or dolime to convert soluble magnesium to Mg(OH)₂. The waste brine (8) is then thermally treated (7) with CO₂. This advantageously results in the above described conversion of Mg(OH)₂ to magnesium carbonate (9). The conversion is generally effective to about 80%. The magnesium carbonate produced is surface dried and processed (11) and used in final the production of products, such as fertiliser. For example, as previously noted, ammonia produced as a by-product during treatment of the gaseous CO₂ (2) may be used as a raw ingredient in fertiliser production.

It is worth reiterating that additional calcium hydroxide produced during the thermal treatment (7), or excess calcium hydroxide added to the waste brine (8) to form Mg(OH)₂, may also be subjected to the carbonation process to form a calcium carbonate product.

Although not illustrated, a potion of the Sc CO₂ may be reacted with Mg(OH)₂, for example in a fluidised bed reactor.

Turning generally to the process for recovery of magnesium metal, unreacted Mg(OH)₂ (10), corresponding to an amount of about 20% of that originally subjected to thermal treatment (7), is calcined (12) at temperature to form MgO. This is then condensed in a plasma furnace (13) to produce Mg metal. As illustrated, CO₂ produced in the plasma furnace (13) may be returned to the thermal treatment (7).

Calcium recovered during processing, for example during pretreatment (5), may be used as a fluxing agent in the plasma furnace (13) if desired.

It will be appreciated that while FIGS. 1 and 2 describe the solution containing soluble magnesium as being derived from a waste stream from a desalination plant, the invention is not so limited. The solution containing soluble magnesium may suitably be derived from other sources, such as saltworks bitterns, saline groundwater, concentrated seawater, and other saline water having an Mg content of generally greater than 1000 ppm.

Referring to FIG. 3, a more detailed description, in the form of a flow chart, of the conversion process from soluble magnesium to magnesium metal is provided. In particular, a solution containing soluble magnesium, such as from a source described in the immediately preceding paragraph, is reacted (30) with Ca(OH)₂, CaO, and possibly calcium magnesium hydroxide (or dolime) to form a slurry containing Mg(OH)₂. The slurry is fed to a thickener and clarifier and separation (31) conducted to produce a thickened slurry. The by-product stream resulting from the separation (31) is concentrated and precipitated (32) to produce a bittern that is rich in sodium and potassium salts and in calcium chloride, which may be recovered if desired. Some gypsum may also be recovered during concentration and precipitation (32).

Dewatering (33) of the thickened slurry is then carried out to produce a filtrate, that may be fed for concentration and precipitation (32), and a Mg(OH)₂ filter cake As will be appreciated from previous discussions, the filter cake may also contain an amount of Ca(OH)₂ if desired.

Calcium carbonate, calcium oxide and/or calcium hydroxide may be optionally blended with the Mg(OH)₂. The Mg(OH)₂, or blend containing it, is then calcined (34) to form MgO (or a precursor oxide blend). CO₂ produced during calcination (34) may be dealt with as desired, for example this may be captured and sold or discharged.

The MgO, or precursor oxide blend, is then blended (35) with either ferro silicon or aluminium oxide, as reductants to the process, and calcium oxide, which is used as a fluxing agent to the process. Depending on the purity of oxide fed to the furnace, 5 tonnes of MgO will produce about one tonne of metal of 99.95% purity. The blends are mixed according to which furnace and feedstock options are selected. Three furnace feed configurations are considered below. They are described under the generic process of DC arc plasma furnaces as metallothermic processing. That is, they use condensation of magnesium vapour as the process for producing magnesium metal.

The present invention suggests the conversion of MgOH₂ as the feedstock for producing magnesium metal. The conventional processes involve conversion of magnesium vapour using magnesite ore or dolomite.

| | Config. 1 | Config. 2 | Config. 3 |
|---|---|---|---|
| Magnesite | N/A | N/A | N/A |
| Dolomite | N/A | N/A | 67% |
| MgOH2 | 70% | 70% | N/A |
| Reductant | FeSi - 20% | FeSi - 20% | Al₂O₃ - 13% |
| | or | or | |
| | Al₂O₃- 13% | Al₂O₃- 13% | |
| Fluxing agent | CaO - 10% | CaO - 10% | CaO - 20% |

Reductants are included on the basis of optional selection of either FeSi (ferro silicon) or aluminium scrap turnings or cuttings. If Al₂O₃ is selected the magnesia input is adjusted accordingly.

The Mg(OH)₂ specification is as follows:

| | |
|---|---|
| MgO | 93-99% |
| CaO | 1-6% |
| Cl | <0.3% |
| Na | <0.1% |
| SO4 | <0.3% |
| Si | <0.7% |
| Fe | <0.3% |
| Al | <0.3% |
| Mn | <0.1% |
| Insolubles | <0.8% |
| Moisture | <1.0% |

Average particle size 3-8 microns.

Another consideration is that of temperature requirement, and therefore energy requirement. FeSi requires more energy and, therefore, the use of Al₂O₃ is considered preferable for use as the reductant.

The oxide blend is then subjected to reduction (36) in a furnace in the presence of aluminium. As previously noted, ferro silicon may also be suitably employed during reduction (36). Reduction (36) may be conducted, for example, at temperatures of approximately 1100° C. for Al₂O₃ (and 1700° C. for FeSi) in an argon sealed DC arc plasma furnace.

Mg metal vapour produced in the furnace may be condensed to liquid Mg metal (37) and/or to solid Mg metal (38). More particularly, the furnace design and configuration of the reaction zone within the furnace causes highly charged particles of magnesium vapour to be directed to an exit port of the furnace. The liquid metal may be refined (39) if necessary and subjected to casting (40). The solid Mg metal may be melted (41) and optional refined, and then cast (42) as desired. Alternatively, the solid Mg metal may be palletised (43).

Likewise, the molten slag produced during reduction (36) in the furnace may be cast (44), or may be cooled and crushed (45) for downstream blending (46).

Referring to FIG. 4, waste derived from coal-fired power plants and desalination plants is substantial, generally due to the extreme quantities of such waste. This presents a globally recognised environmental problem insofar as schedules for the treatment of such high throughput waste streams are relatively difficult to devise. The present invention, at least in certain aspects, aims to utilise carbon dioxide generated during the burning of coal as a feed material to facilitate recovery of magnesium carbonate from a high throughput stream containing soluble magnesium derived from a desalination plant.

The magnesium carbonate product is a commodity that may be put to use in a number of industries, including direct use in the chemical industry, use in fertiliser production, and use in agri-water industries. As will be appreciated from the above description of the invention, magnesium metal is also a valuable product of the process of the invention.

It will of course be realised that the above has been given only by way of illustrative example of the invention and that all such modifications and variations thereto as would be apparent to those of skill in the art are deemed to fall within the broad scope and ambit of the invention as herein set forth.

1. A process for the recovery of magnesium from a solution containing soluble magnesium, the process comprising:
precipitating magnesium hydroxide from the solution;
forming an oxide blend including magnesium oxide derived from the precipitated magnesium hydroxide together with calcium oxide;
reducing the oxide blend to form a magnesium metal vapour; and
condensing the vapour to recover magnesium metal.

2. A process according to claim 1, wherein precipitation of the magnesium hydroxide includes treatment of the solution containing soluble magnesium with at least one of, the group consisting of sodium hydroxide, calcium oxide, calcium magnesium oxide and calcium hydroxide.

3. A process according to claim 2, wherein the solution containing soluble magnesium is treated with calcium oxide and/or calcium magnesium oxide to separately recover magnesium hydroxide and calcium hydroxide.

4. A process according to claim 3, wherein magnesium hydroxide and calcium hydroxide are recombined and calcined to form the oxide blend.

5. A process according to claim 2, wherein the solution containing soluble magnesium is treated with sodium hydroxide derived from the solution prior to precipitation of the magnesium hydroxide.

6. A process according to claim 5, wherein prior to the precipitation of magnesium hydroxide the solution containing soluble magnesium is treated to remove sodium chloride which is converted to sodium hydroxide for use in said precipitation of magnesium hydroxide.

7. A process according to claim 6, wherein the solution containing soluble magnesium is subjected to electrodialysis to remove sodium chloride.

8. A process according to claim 7, wherein the sodium chloride removed is electrolysed to form sodium hydroxide.

9. A process according to claim 1, wherein a reductant is added to the oxide blend prior to reduction.

10. A process according to claim 9, wherein the reductant is ferro silicon or aluminium oxide.

11. A process according to claim 1, wherein aluminium metal is added to the oxide blend before and/or during the reduction process.

12. A process according to claim 1, wherein the oxide blend is reduced in a plasma DC Arc furnace to produce magnesium metal vapour.

13. A process according to claim 12, wherein the temperature is maintained at from 1100° C. to 1700° C. during the reduction process.

14. A process according to claim 1, wherein a portion of the magnesium hydroxide is converted to magnesium carbonate in a carbon dioxide sequestration process.

15. A process according to claim 14, wherein the magnesium hydroxide is reacted with the carbon dioxide in a fluidised bed reactor.

16. A process according to claim 1, including recovering sodium chloride from the solution containing soluble magnesium and reporting the sodium chloride to a bioreactor housing microalgae for the production of biomass oil.

17. A process according to claim 16, including reporting carbon dioxide produced during processing to the bioreactor.

18. A process according to claim 16, wherein the solution containing soluble magnesium is treated using electrodialysis to produce a plurality of streams having different salinity that are each fed to separate bioreactors, each bioreactor housing microalgae.

19. A process for the recovery of magnesium from a waste stream of a desalination plant, the process comprising:
treating the waste stream using electrodialysis to remove sodium chloride;
converting the sodium chloride removed from the waste stream to sodium hydroxide;
reacting the sodium hydroxide with soluble magnesium in the waste stream to precipitate magnesium hydroxide;
forming an oxide blend including magnesium oxide derived from the precipitated magnesium hydroxide together with calcium oxide;
reducing the oxide blend to form magnesium metal vapour;
condensing the magnesium metal vapour to recover magnesium metal; and
reporting at least a portion of the sodium chloride removed from the waste stream and carbon dioxide to a bioreactor housing microalgae for the production of biofuel.

20. A method for treatment of a waste stream emitted from a desalination plant comprising:
treating the waste stream to remove sodium chloride;
converting the sodium chloride removed from the waste stream to sodium hydroxide; and
reacting at least a portion of the sodium hydroxide with soluble magnesium in the waste stream to precipitate magnesium hydroxide.

21. A process according to claim 20, including reporting at least a portion of the sodium hydroxide back to the waste stream and/or to an outlet for the waste stream thereby modulating the pH of the water at the outlet for the waste stream.
